(19) United States
(12) Patent Application Publication
(10) Pub. No.: US 2022/0280260 A1
(43) Pub. Date: Sep. 8, 2022

(54) MARKING DEVICE AND METHOD

(71) Applicant: 1147065 B.C. Ltd., Vancouver (CA)

(72) Inventors: Case Bryant VAN WYNGAARDEN, Vancouver (CA); Mark Stephen SEGAL, Vancouver (CA); Sanjiv Bhalla BHALLA, Vancouver (CA); Patrick Michael HANEY, Vancouver (CA); Christopher Sandor POLLOCK, Vancouver (CA); Peter Andrew ZAKRZEWSKI, Vancouver (CA); Colin NYULI, Vancouver (CA); Alfred CHAU, Vancouver (CA); Darren Addison GRAY, Vancouver (CA)

(21) Appl. No.: 17/631,346

(22) PCT Filed: Jul. 29, 2020
(86) PCT No.: PCT/CA2020/051039

§ 371 Date: Jan. 28, 2022

Related U.S. Application Data
(60) Provisional application No. 62/879,985, filed on Jul. 29, 2019.

(51) Int. Cl. A61B 90/00

(52) U.S. Cl. CPC A61B 90/39; A61B 2090/395; A61B 2090/3962

(57) ABSTRACT

A medical marking device can produce visible temporary marks, including at least one of indentations or colorations, in a specified pattern on skin or tissue of a patient. The marking device can include a body, such as a cap, that can removably attach over an end of a surgical marker. The body can include marking features, such as blades, optionally positioned at a longitudinal end of the body. The marking features can be pressed against the skin or tissue of the patient to form the visible temporary marks. The marking features can optionally be inked, optionally by the surgical marker, before the marks are formed. The marking features can optionally be distributed between a first subset, positioned generally along a line, and a second subset, positioned asymmetrically with respect to the line. An optional inclinometer can attach to the body to allow alignment to a horizontal direction.

### FIELD OF THE DISCLOSURE

The present disclosure relates generally to marking of a patient's skin or tissue, during or before a medical procedure.

### BACKGROUND OF THE DISCLOSURE

Some medical procedures can include marking the skin or tissue of a patient. In some examples, a practitioner can mark the skin or tissue so that the practitioner can return to the marked location in a later part of a procedure. In some examples, the practitioner can use the marking to show a proper orientation for a surgical tool or device.

For example, in an ultrasound guided procedure, a practitioner can identify a target location by scanning portions of the patient's anatomy with an ultrasound transducer. The ultrasound transducer can contact the patient's skin through ultrasound gel. When the practitioner has determined the target location, the practitioner can mark a needle entry point on the patient's skin, and the skin can then be sterilized.

Marking the needle entry point is challenging, due to the presence of the ultrasound gel. Using a surgical pen to form the markings is not ideal, because pen ink may not mark clearly through the ultrasound gel, and the ink can further smudge or be removed entirely upon sterilizing the skin. Some practitioners attempt to form an imprint on the skin or tissue using a needle cap or a retracted ball point pen. These imprints can be of poor quality and may be inaccurate.

As a result, these inadequate current marking techniques can lead to poor orientation of a probe, which leads to increased time to orient the ultrasound probe during the sterile portion of the procedure. Further, these inadequate current marking techniques can often require remarking, which can add time and expense to a procedure.

For example, for some procedures, it can be beneficial to mark an orientation of a transducer as well as a needle entry site. For example, in some surgical procedures, a practitioner can rely on a particular pattern marked on the skin, which can show an orientation in addition to a location. Typically, the practitioner draws such a pattern freehand with a surgical pen.

As another example, in a toric intraocular lens implantation procedure, a practitioner can identify a suitable rotational orientation for the implantable lens by marking the patient's cornea. For corneal marking, a practitioner typically applies ink to a metal tool by rubbing the tool with a standard surgical marker, then marks the cornea with the inked tool. The metal tool requires sterilization following each use. In general, marking the cornea in this manner can be cumbersome, due to the use of multiple tools, and expensive.

Accordingly, there exists a need for a device and method that can form improved markings on the patient's skin or tissue.

### SUMMARY

A marking device can produce visible temporary marks, including at least one of indentations or colorations, in a specified pattern on skin or tissue of a patient. The marking device can include a body, such as a cap, that can removably attach over an end of a surgical marker. The body can include marking features, such as blades, optionally positioned at a longitudinal end of the body. The marking features can be pressed against the skin or tissue of the patient to form the visible temporary marks. The marking features can optionally be inked, optionally by the surgical marker, before the marks are formed. The marking features can optionally be distributed between a first subset, positioned generally along a line, and a second subset, positioned asymmetrically with respect to the line. The subsets may optionally also create markings at the end points of a profile which collectively define the profile. An optional inclinometer can attach to the body to allow alignment to a horizontal direction.

### BRIEF DESCRIPTION OF THE DRAWINGS

FIG. 1 shows a perspective view of an example of a marking device with a body that can function as a pen cap, in accordance with some examples.

FIG. 2 shows a front view of the marking face of the marking device of FIG. 1, in accordance with some examples.

FIG. 3 shows a perspective view of another example of a marking device, in accordance with some examples.

FIG. 4 shows an exploded view of an example of a device cap configured to mate with the marking device of FIG. 3, in accordance with some examples.

FIG. 5 shows a perspective view of another example of a marking device with a body that can function as a pen cap, in accordance with some examples.

FIG. 6 shows a front view of an example of a marking device for generating markings on a patient's eye, in accordance with some examples.

FIG. 7 shows a perspective view of the marking device of FIG. 6, in accordance with some examples.

FIG. 8 shows a perspective view of another example of a marking device, in accordance with some examples.

FIG. 9 shows a front view of the marking device of FIG. 8, in accordance with some examples.

FIGS. 10-12 show an example of a technique for marking the skin, using the marking device of FIG. 1. In FIG. 10, the marking device is to be advanced toward a specified location on the patient's body. In FIG. 11, the marking device is compressed into the patient's body. In FIG. 12, the marking device has been retracted from the patient's body, with a resulting mark having been formed where the marking device contacted the patient's body.

FIG. 13 shows a front view of another example of a marking device having equally spaced graduations on a planar face allowing the cap to be used as a ruler, in accordance with some examples.

Corresponding reference characters indicate corresponding parts throughout the several views. Elements in the drawings are not necessarily drawn to scale. The configurations shown in the drawings are merely examples, and should not be construed as limiting the scope of the invention in any manner.

### DETAILED DESCRIPTION

An improved surgical marking device and method is discussed herein. The device can form improved markings on the skin or tissue of a patient.

Known devices are discussed in U.S. Pat. No. 6,805,669, United States Patent Application No. 2006/0106312, and United States Patent Application No. 2012/0209280.

The present examples of a device and method can improve over these and other known marking techniques. One improvement can include indicating a direction or orientation, in addition to a location on the skin or tissue. Another improvement can include an increased ability to remain on the skin or tissue after the skin or tissue has been sterilized. Another improvement can include a decreased cost, compared to some typical marking devices. Another improvement can be an inclusion of the marking ink and the marking component (e.g., the part that forms an imprint on the skin or tissue) on the same device.

In some examples, the device discussed herein can be formed as a pen cap, which can couple with a surgical marking pen. In some examples, the pen cap can include a marking face with a profile outlined by one or more marking tines, bevels, blades, or other marking features. In some examples, the pen cap can include a cavity that can removably couple onto an end of a standard surgical marking pen, and thus the pen can serve as a handle for the operator to grasp and use to manipulate the marking device.

The marking device has marking features that produce temporary marks in a specified pattern. The pattern of the temporary marks can provide guidance and information. In some examples, the profile can provide information to the practitioner. For example, a “T” shaped profile can provide the practitioner with information regarding transducer orientation and an axis for needle access in ultrasound-guided procedures. Additionally, the resulting marking points can optionally be equally spaced at a standard distance, providing a measurement reference for the practitioner.

In some examples, the marking caused by the device and method discussed herein can be a temporary skin indentation, optionally coupled with ink to form an inked indentation. There are several techniques to couple the ink to the marking features of the device. In one example, the practitioner can rub a skin marker against the marking features of the device, to transfer ink from the skin marker to the marking features. In other examples, the marking features can be manufactured with ink pre-coated on the marking features or present in channels within the marking features. In still other examples, the device can include an absorbent pad positioned at the base of the cap, with the absorbent pad including ink. In these examples, the absorbent pad can transfer ink to the marking features by contacting the marking features when the cap is placed over the marking features.

In some examples, which can form markings on a cornea for toric intraocular lens implantation procedures, the device can form markings at one or more of the 0°, 90°, 180° or 270° axes of the cornea. These angles are not intended to be limiting and any angle may be marked. In some examples, the marking features can optionally be rotatable relative to an inclinometer, allowing for the practitioner to implant the intraocular lens at a desired angle of marking relative to a horizontal meridian of the eye. Angular marking of the eye is useful in toric intraocular lens procedures for aligning features of the implant with the steep axis of astigmatism of the eye.

In some examples, the pen cap can optionally include a flat face with equally spaced graduations, for use as a ruler in measuring features on the skin or tissue of the patient.

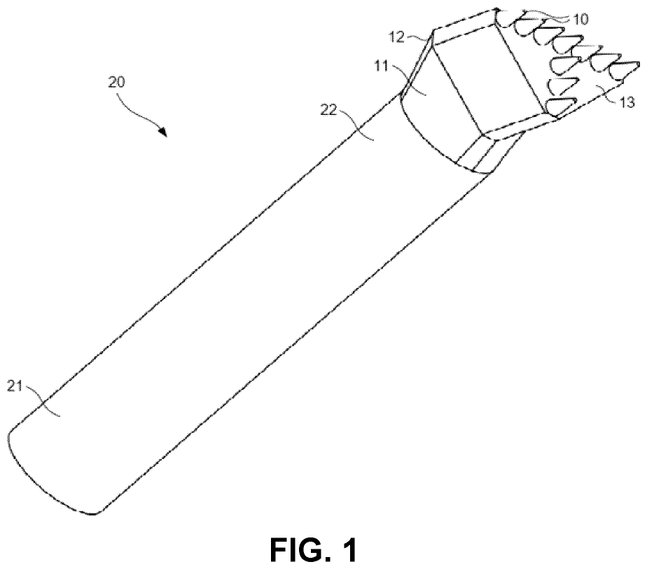

FIG. 1 shows a perspective view of an example of a marking device with a body that can function as a pen cap, in accordance with some examples. In some examples, a practitioner can use the marking device of FIG. 1 to generate markings on a patient's skin or any other tissue prior to an ultrasound-guided procedure.

A body section 20 can include a proximal section 21. In some examples, the proximal section 21 can include a standard pen cap geometry for mating with a surgical pen, which can then serve as a handle which allows the operator to grasp and manipulate the marking device. The body section 20 can include a distal section 22. In some examples, the distal section 22 can be continuous with a transition section 11 from the body to the marking face 13. The transition section 11 may be continuous with an angled junction 12, which may be continuous with the marking face 13. Protruding from the marking face 13 are protrusions such as conical marking features 10 which create the mark in skin when depressed into the patient's body without piercing or damaging the tissue. The marking features 10 can optionally form a “T” pattern, which can provide ultrasound transducer orientation and needle axis orientation for ultrasound-guided procedures. The marking features 10 can optionally be partially or entirely composed of radiopaque material for visualization under fluoroscopy.

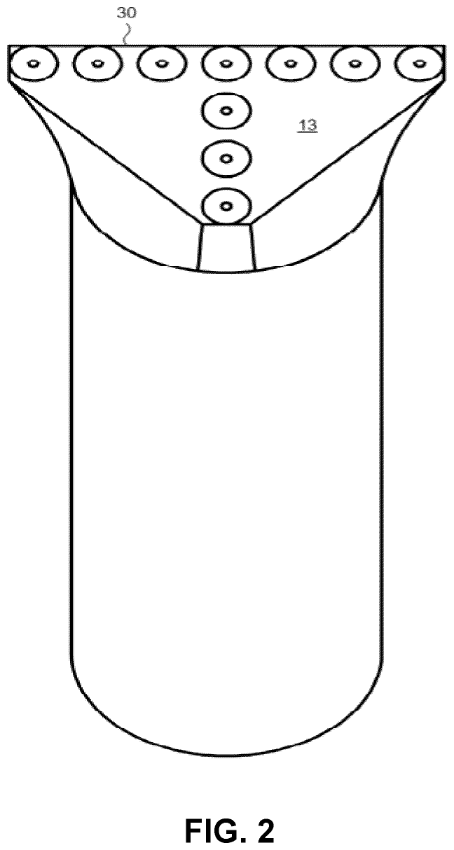

FIG. 2 shows a front view of the marking face 13 of the marking device of FIG. 1, in accordance with some examples.

In some examples, the marking face 13 can optionally include a flat edge 30, which can optionally sit flush against an ultrasound transducer.

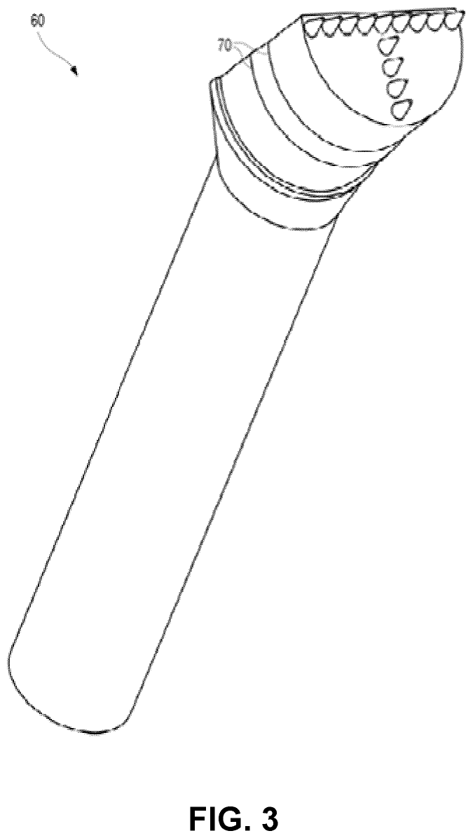

FIG. 3 shows a perspective view of another example of a marking device, in accordance with some examples.

In the example of FIG. 3, the marking device is continuous with the device head 60. Compared with the example in FIG. 1, the marking device of FIG. 3 additionally includes optional various sized radii 70 for mating with a device cap for capping of the marking face.

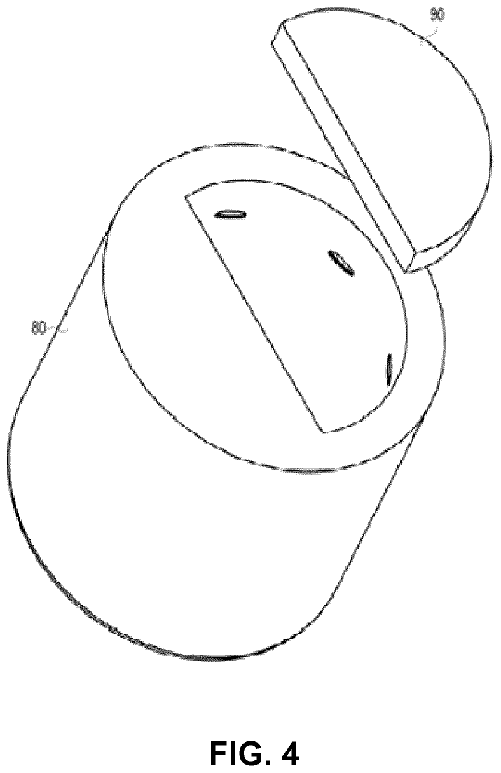

FIG. 4 shows an exploded view of an example of a device cap 80 configured to mate with the marking device of FIG. 3, in accordance with some examples.

In some examples, the device cap 80 can include an absorbent pad 90 positioned at a base of a device cap channel. In some examples, the absorbent pad 90 can optionally include a skin marking solution. In some examples, the marking features of the marking device of FIG. 3 can optionally rest against the absorbent pad 90 when the marking device of FIG. 3 is capped with the device cap of FIG. 4.

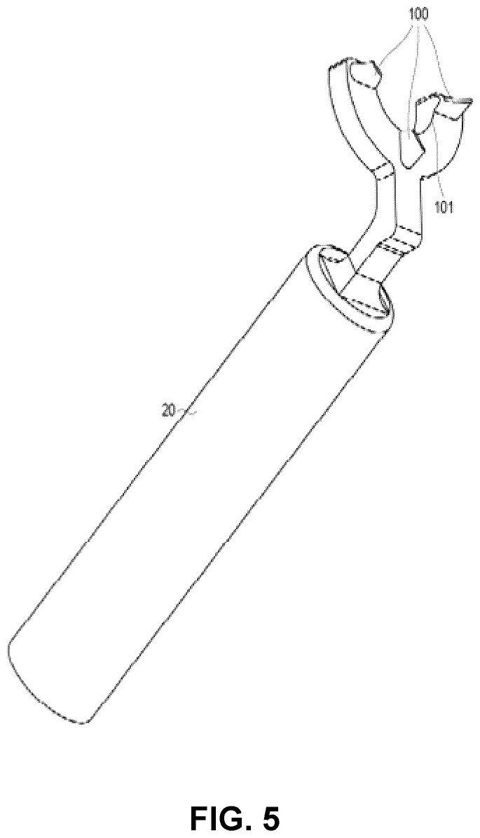

FIG. 5 shows a perspective view of another example of a marking device with a body that can function as a pen cap, in accordance with some examples. In some examples, a practitioner can use the marking device of FIG. 5 to generate markings on a patient's eye for use in toric intraocular lens implantation.

In some examples, the marking face 101 can optionally include a partial circle with a gap in the center (or a complete circle) for viewing of the patient's eye. The gap may be a semi-circular shape. In some examples, the marking features can optionally include one or more curved surfaces 100. In some examples, the one or more curved surfaces 100 can optionally have a radius of curvature similar to that of a human eye. In other examples, the marking features can optionally include one or more other surface geometries, such as tines or bevels.

The marking features can optionally include one or more curved surfaces 100 to complement different shaped surfaces to mark. The marking features contact the curved surface with similar amount of pressure.

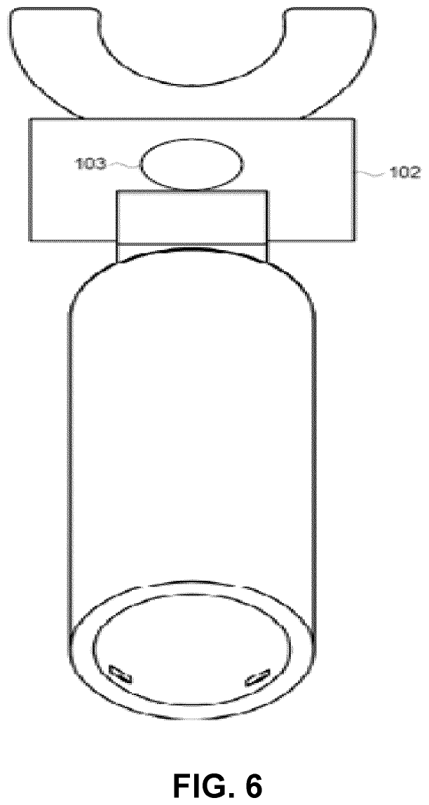

FIG. 6 shows a front view of an example of a marking device for generating markings on a patient's eye, in accordance with some examples.

In the example of FIG. 6, the marking device can optionally include a bubble level, which can function as an inclinometer. In some examples, the bubble level can include a transparent cylindrical housing 102. The housing 102 can include a liquid and an air bubble 103.

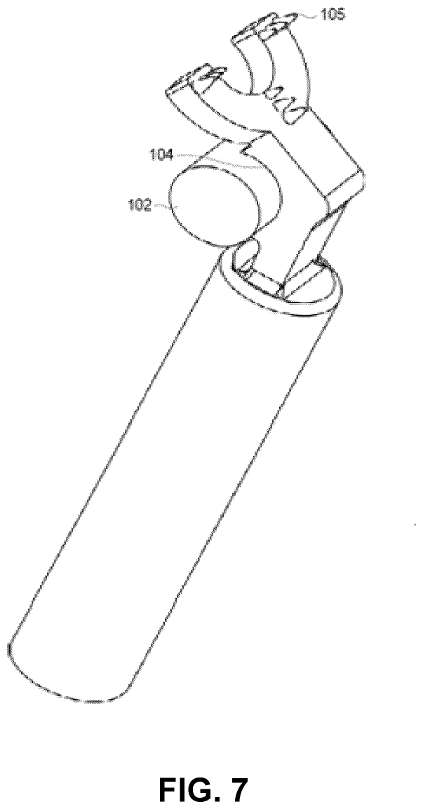

FIG. 7 shows a perspective view of the marking device of FIG. 6, in accordance with some examples.

In some examples, the device can include an optional snap fit feature 104, which can removably or fixedly attach to the housing 102 (e.g., the inclinometer). Other coupling mechanisms known in the art may also be used to joint the device and inclinometer together such as fasteners including pins, screws, hook and loop, and others

Compared to the example in FIG. 3, the device of FIG. 7 can optionally additionally include marking tines 105 having different heights, so that when pressed against the cornea of a patient, the marking tines 105 can accommodate the curved surface of the cornea.

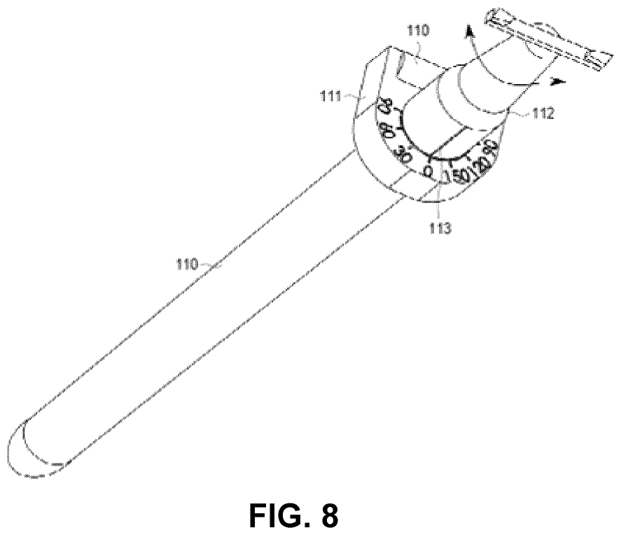

FIG. 8 shows a perspective view of another example of a marking device, in accordance with some examples.

The marking device of FIG. 8 can include marking features that are rotatable relative to an inclinometer, for marking the cornea at specifiable angles relative to a horizontal meridian of the eye. An angular alignment indicator 113, optionally formed as a slot or a line on a rotatable marking cap 112, can be aligned with a desired angular marking on the inclinometer holder 111.

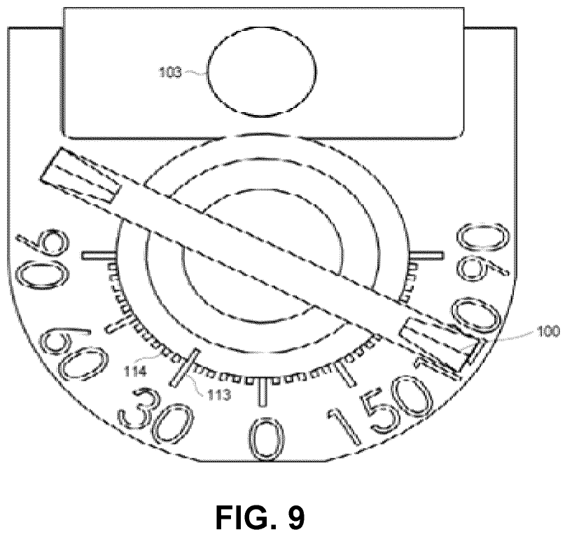

FIG. 9 shows a front view of the marking device of FIG. 8, in accordance with some examples.

In the configuration shown in FIG. 9, the marking features 100 have been rotated to mark the eye at 30° relative to the horizontal meridian. This 30° angle is achieved by aligning the angular indicator 113 on the marking face with the angular markings 114 on the inclinometer holder. The numerical example of 30° is but one example; other suitable numerical values can also be used.

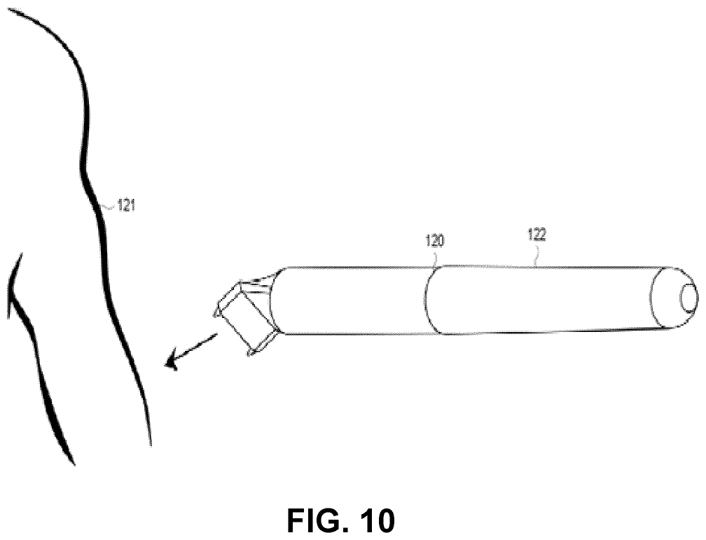

FIGS. 10-12 show an example of a technique for marking the skin, using the marking device of FIG. 1. In the marking device of FIG. 1, the marking device is formed as a pen cap, and is coupled with a standard surgical marker. The proximal end of the marking device can accept the distal end of the surgical ben body 122. The surgical pen and the marking device can meet at the mating edge 120. Such a technique can also use any of the other marking device configurations shown and discussed herein, as well as any suitable marking devices that are not shown and discussed herein.

In FIG. 10, the marking device is to be advanced toward a specified location on the patient's body. In this example, the specified location is on the patient's forearm. Other suitable locations can also be used.

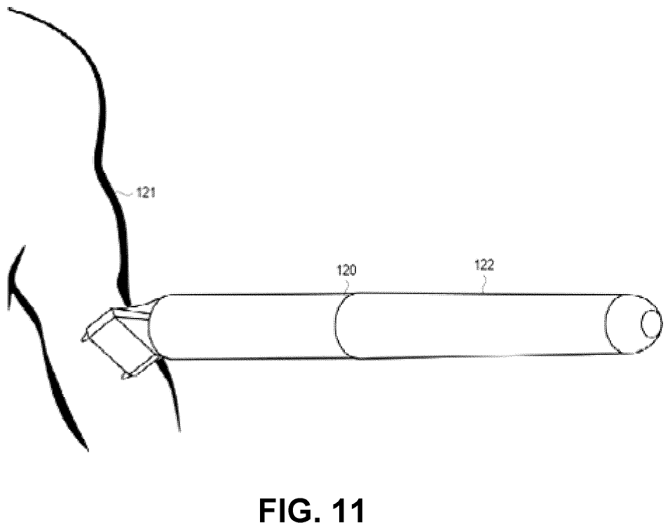

In FIG. 11, the marking device is compressed into the patient's body 121. During use, the practitioner can grip the marking device anywhere on the surgical pen body or on the marking device body. During use, the marking face can be generally orthogonal to the patient's body when the marking features are compressed into the patient's body.

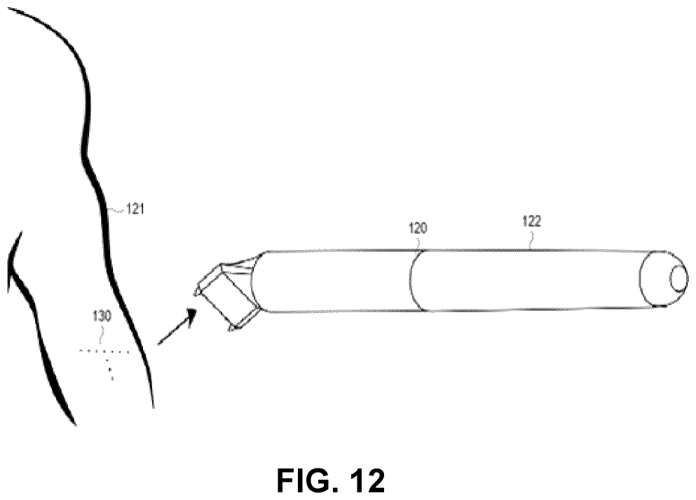

In FIG. 12, the marking device has been retracted from the patient's body, with a resulting mark 130 having been formed where the marking device contacted the patient's body. In the example of FIG. 12, the marking pattern 130 can provide ultrasound transducer orientation and needle access axis. Other suitable patterns can also be used.

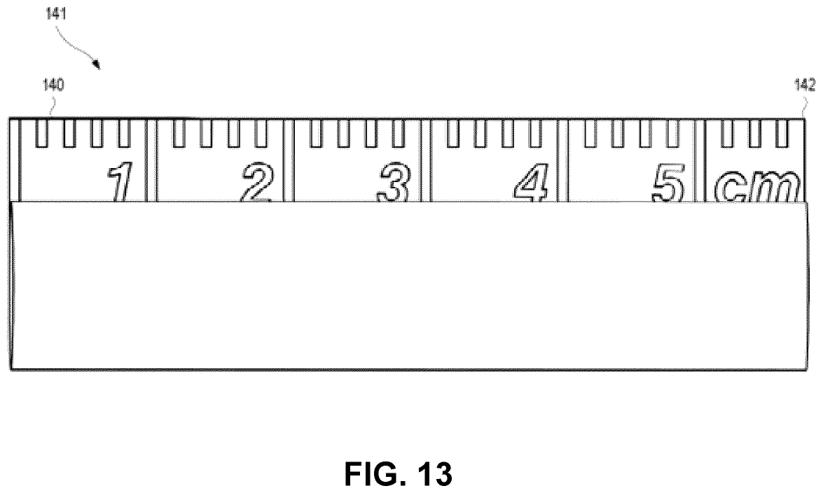

FIG. 13 shows a front view of another example of a marking device having equally spaced graduations 140 on a planar face 141 allowing the cap to be used as a ruler, in accordance with some examples. The planar face can have a straight edge 142 which may be held against the patient for assistance in drawing straight lines. The ruler may be used with any of the examples disclosed herein.

Embodiments described herein provide a marking device to produce visible temporary marks in a specified pattern on skin or tissue of a patient. The marking device can include a body, such as a cap, that can removably attach over an end of a surgical marker. The body can include marking features, such as blades, optionally positioned at a longitudinal end of the body. The marking features can be pressed against the skin or tissue of the patient to form the visible temporary marks. The marking features can optionally be inked, optionally by the surgical marker, before the marks are formed. The marks can be different indentations or colorations.

In some examples, the marking features are generally conical blades having tips that are positioned in a same plane. The conical blades can extend from a generally planar surface. The marking features can be planar if the tissue surface they are marking is close to planar locally (i.e. most parts of the skin). An example pattern in the “T” for ultrasound and the line for the eye.

In some examples, the marking features are generally conical blades having tips that are positioned in different planes. The marking features on different planes can be used for marking distinctly curved surfaces like the eye.

In some examples, the blades have a same depth. In other examples, at least two of the blades have different depths. The varying depths and non-planar features can be for contouring to a curved surface.

In some examples, the marking features can be grouped in different sets to define the boundary of the resulting marks. By grouping into sets there can be fewer marks, but still define the necessary geometry of the mark. For instance, if a mark needs to be a line, there could be grouped sets at each end of the line that define the line

In some examples, the marking features can optionally be distributed between a first subset, positioned generally along a line, and a second subset, positioned asymmetrically with respect to the line. The subsets may optionally also create markings at the end points of a profile which collectively define the profile.

There can be a first subset of the marking features positioned generally along a line, and a second subset of the marking features positioned asymmetrically with respect to the line. In some examples, at least some of the first subset of the marking features are evenly spaced along the line.

In some examples, the first subset of the marking features are distributed between a third subset of the marking features and a fourth subset of the marking features. The third subset and the fourth subset can be separated by a gap sized to accommodate a cornea of a human eye. An optional inclinometer can attach to the body to allow alignment to a horizontal direction.

What is claimed is:

1. A medical marking device, comprising:
a body configured to removably attach to a surgical marker,
the body including a plurality of marking features that are configured to produce visible temporary marks in a specified pattern on skin or tissue of a patient when the plurality of marking features are pressed against the skin or tissue of the patient.

2. The medical marking device of claim 1, wherein:
the body is configured as a cap with a central channel;
the cap is removably attachable over an end of the surgical marker; and
the marking features are positioned at a longitudinal end of the cap.

3. The medical marking device of claim 2, further comprising an ink pad positioned to contact the marking features when the surgical marker is capped.

4. The medical marking device of claim 1, wherein the visible temporary marks include indentations arranged in the specified pattern.

5. The medical marking device of claim 4, wherein:
the marking features are further configured to accept ink from the surgical marker when the marking features are pressed against a tip of the surgical marker; and
the marking features are further configured to deposit the accepted ink in the specified pattern on the skin or tissue of the patient, such that the visible temporary marks further include colorations with the indentations.

6. The medical marking device of claim 1, wherein the marking features are generally conical blades having tips that are positioned in a same plane.

7. The medical marking device of claim 1, wherein:
the marking features are generally conical blades having tips that are positioned along a curved surface; and
the curved surface is convex when viewed from a handle of the body.

8. The medical marking device of claim 1, wherein:
the marking features are generally conical blades extending from a generally planar surface; and
the blades have a same depth.

9. The medical marking device of claim 1, wherein:
the marking features are generally conical blades extending from a generally planar surface; and
at least two of the blades have different depths.

10. The medical marking device of claim 9, wherein the blades have depths that increase with distance away from a center of the specified pattern.

11. The medical marking device of claim 1, wherein a first subset of the marking features are positioned generally along a line, and a second subset of the marking features are positioned asymmetrically with respect to the line.

12. The medical marking device of claim 11, wherein at least some of the first subset of the marking features are evenly spaced along the line.

13. The medical marking device of claim 11, wherein:
the first subset of the marking features are distributed between a third subset of the marking features and a fourth subset of the marking features;
the third subset and the fourth subset are separated by a gap;
the gap sized to accommodate a cornea of a human eye.

14. The medical marking device of claim 1, further comprising an inclinometer oriented generally orthogonal to a longitudinal axis of the body and configured to align the body such that the inclinometer extends along a horizontal orientation.

15. The medical marking device of claim 14, wherein the inclinometer is removably attachable to the body.

16. The medical marking device of claim 14, further comprising a rotator configured to rotate the marking features by a specified angle with respect to the lateral line.

17. The medical marking device of claim 1, wherein at least some of the marking features include a radiopaque material.

18. The medical marking device of claim 1, wherein the body includes at least one substantially straight edge and graduations positioned along the substantially straight edge.

19. A method for marking tissue, comprising:
removing a cap from a surgical marker, the cap including a plurality of marking features positioned in a specified pattern at a longitudinal end of the cap;
bringing the marking features into contact with a tip of the surgical marker to coat the marking features with ink; and
pressing the ink-coated marking features against skin or tissue of the patient to form visible temporary indentations in the specified pattern on the skin or tissue of the patient and colorations arranged in the specified pattern on the skin or tissue of the patient.

20. A medical marking system, comprising:
a cap configured to removably attach to a surgical marker over an end of the surgical marker,
the cap including a plurality of blades positioned in a specified pattern at a longitudinal end of the cap,
the blades being distributed between a first subset of the plurality of blades and a second subset of the plurality of blades,
the first subset of the blades being positioned generally along a line,
the second subset of the blades being positioned on one side of the line.
